(19) United States
(12) Patent Application Publication
(10) Pub. No.: US 2007/0009490 A1
(43) Pub. Date: Jan. 11, 2007

(54) Dried biotherapeutic composition, uses, and device and methods for administration thereof

(76) Inventors: Anthony E. Conte, Dix Hills, NY (US); David C. Katz, Morris Plains, NJ (US); Richard L. Dillingham, Minneapolis, MN (US)

Correspondence Address: Martin D. Moynihan;PRTSI, Inc., P.O. Box 16446, Arlington, VA, 22215, US

(21) Appl. No.: 11/392,654

(22) Filed: Mar. 30, 2006

Related U.S. Application Data
(63) Continuation-in-part of application No. 10/953,770, filed on Sep. 30, 2004.
(60) Provisional application No. 60/507,503, filed on Oct. 2, 2003.

(51) Int. Cl. A61K 35/74; C12N 1/20; A61M 31/00

(52) U.S. Cl. 424/93.4; 435/252.1; 604/500

(57) ABSTRACT

A biotherapeutic composition containing rapidly activatable bacteria in a dry form, a device for administering such a composition and methods of treatment thereof are disclosed. A method for preparing the biotherapeutic composition itself, as well as a method for preparing the bacteria for such a composition is also disclosed.

### RELATED APPLICATIONS

The present application is a continuation-in-part of U.S. patent application Ser. No. 10/953,770, filed on 30 Sept. 2004, which claims the benefit of U.S. Provisional Patent Application No. 60/507,503, filed on 2 Oct. 2003, the contents of which are incorporated herein by reference.

### FIELD OF THE INVENTION

The present invention relates to biotherapeutic compositions, and more particularly to a dried biotherapeutic composition comprising a non-pathogenic bacterial strain, as well as uses, compositions, methods of treatment, and device and methods for administration thereof.

### BACKGROUND OF THE INVENTION

Probiotic bacteria are those which are beneficial to humans and/or animals. The use of probiotic bacteria is known in the art for improving the microbial balance in the intestinal tract of mammals, in order to prevent or treat gastro-enteric infections and other diseases or disorders involving and/or causing changes in or to the intestinal microflora composition, and/or resulting in any change to the microflora composition, and/or maintaining such changes, as well as changes to the microflora composition which actively cause or potentiate such diseases or disorders.

However, the results of studies carried out to date have been inconsistent and/or ambiguous. For example, in some studies, the use of probiotic bacteria alone to treat “traveler's diarrhea” was not sufficient to provide a significant effect in patients as compared to place, yet the combination of the probiotic treatment with antibiotics proved to be highly effective. Other studies have shown that probiotic treatment alone has a beneficial effect, yet such an effect often required 3-6 months to become apparent (see also, for example, J. JAMA, 1996, vol. 275, No 11; and U.S. Pat. Nos. 5,433,826 and 5,589,168).

Recent studies have been directed towards investigation of the effects of various types of probiotic bacteria, either alone or in combination; improvement of the survival rate of probiotic bacteria and methods of enabling long-term preservation; biomass accumulation, and the use of probiotic bacteria in prophylaxis and treatment of humans and animals.

Approximately 400 different kinds of bacteria and bacteroids are known to exist in the digestive tract of humans and other mammals, which may provide about 30-40% of excrement volume. The characteristics and functions of only about 15 of these known types have been studied in any detail.

Each of these types of bacteria occupies its own ecological niche in the digestive tract, each having particular conditions for optimal survival and multiplication rate.

Pathogenic bacteria, which may cause various diseases or disorders, also occupy their own particular environmental niches or habitats. Competition between pathogenic and probiotic bacteria may occur under various conditions, but maximal competitive effect occurs when the conditions for optimal survival and multiplication rate of both pathogenic and probiotic bacteria are similar. Under such conditions, survival depends upon more stringent competition for nutrients or growth factors, as well as upon synergistic nutrient utilization and competition for receptor sites. Factors such as production of antimicrobial substances, intensity of multiplication, and creation of restrictive environment, including induction of immunological processes and stimulation of epithelial cell turnover also have great significance under such conditions.

Probiotic compositions have been developed using cultures of non-pathogenic E. coli with other non-pathogenic bacteria (U.S. Pat. Nos. 5,340,577; 5,443,826; 5,478,557; and 5,604,127).

Lactose-positive non-pathogenic E. coli strains having high antagonistic activity have been produced as freeze-dried preparations in Germany and Russia (e.g. use of freeze-dried preparation Colibacterin siccum of E. coli M17, described in Vidal Handbook: Pharmaceutical preparations in Russia, Astra Pharm Service, 1997, Moscow).

Studies have been carried out using Lactobacteria, which are dried and incorporated into tiny capsules (U.S. Pat. Nos. 5,501,857; 5,614209; and 5,635,202). The authors claim that such a microencapsulated preparation has greater stability than conventional forms during passage through the stomach.

Studies in preservation of living bacteria have largely been directed towards freeze-dried preparations, with regard to improved production methods and technical solutions for simplifying their application (U.S. Pat. Nos. 5,139,792 and 5,401,501).

None of the background art teaches or suggests a probiotic composition in which the bacterial cells are dried (for example by being freeze dried or lyophilized), yet are rapidly able to “reanimate” or achieve a high level of biological activity when brought into contact with the gastrointestinal tract of a subject. Indeed, such dried compositions are known to produce bacteria of inferior quality as a probiotic treatment, because the bacteria either are not capable of growth and/or other biological activities upon entry to the gastrointestinal tract of the subject, or only slowly return to a state of being capable of such growth and/or other biological activities.

Such a readily activatable probiotic composition is clearly needed, for example for such diseases as inflammatory bowel disease.

Inflammatory bowel disease, or IBD, is a collective term encompassing related, but distinct, chronic inflammatory disorders of the gastrointestinal tract, such as Crohn's disease, ulcerative colitis (UC), indeterminate colitis, microscopic colitis and collagenous colitis, with Crohn's disease and ulcerative colitis being the most common diseases. Another chronic disorder of the gastrointestinal tract is irritable bowel syndrome (IBS).

For most patients, IBD and IBS are chronic conditions with symptoms lasting for months to years. They are most common in young adults, but can occur at any age. These conditions occur worldwide, but are most common in industrialized countries such as the United States, England, and northern Europe. For example, IBD affects an estimated one million people in the United States and an equal number in Western Europe.

The exact causes of IBD and IBS are not yet understood. Common hypotheses include, for example, disorders in the immune system and actions of pro-inflammatory cytokines and selective activation of lymphocyte subsets, which perpetuate unrestrained activation of an inflammatory response in the intestine. Metabolites generated by pathogenic and potentially pathogenic bacteria may cause disorders in the immune system. Hence, these bacteria may be implicated in disturbances of this nature, related to disturbances in the microbiological balance in the intestine. Such disturbances may themselves be a cause, or alternatively (or in combination), it is believed that the disturbance may in turn lead to auto-immune reactions and/or other reactions of the immune system. For example, it was recently shown that in patients suffering from IBS, 80% of such patients have bacterial overgrowth in the intestinal system; treatment of this overgrowth led to a reduction or even cessation of symptoms in many patients with IBS (from research by Dr. Mark Pimentel at Cedars-Sinai Medical Center in California).

IBD and IBS have no cure. Patients afflicted with IBD or IBS are currently generally treated with therapies that are directed at reducing the inflammatory processes, and at reducing the effects of the inflammatory processes on the patient. The presently known medical treatment of IBD is intended to decrease the number, frequency and severity of acute exacerbations of inflammatory bowel disease and to prevent secondary complications, but at best, the results are disappointing.

The presently known methods for treating IBD or IBS may fail to provide a solution for at least some IBD or IBS sufferers, as these methods (i) fail to provide a substantial cure for IBD, but rather provide treatment of the symptoms; and (ii) include either drug therapy that is accompanied by severe adverse side effects, or invasive surgical treatments, both affecting the sufferer's quality of life.

Other diseases involving the gastrointestinal tract for which the cause is unknown and/or the treatment is unsatisfactory, include microscopic or lymphocytic colitis and coliagenous colitis, which may represent variants of the same disease. The disease is characterized by a waxing and waning watery diarrhea that usually affects middle-aged females. Colonoscopy shows normal appearance of the mucosa, but biopsy shows infiltration of the lamina propria with inflammatory cells and intraepithelial lymphocytes. It is only in collagenous colitis that a subepithelial band of collagen is present. The pathogenesis of the disorder remains a mystery, but there is evidence, much like UC and Crohn's disease, that the inflammatory process may be triggered by a luminal agent. The disease is treated much like IBD, with 5-amino salicylic acid (5-ASA) drugs and corticosteroid. 5-ASA products may cause headache, nausea, fatigue, abdominal pain and worsening diarrhea. Hypersensitivity reactions may lead to rash, fever, hepatitis, pneumonitis, hemolytic anemia, and bone marrow suppression. Long-term use of corticosteroids may cause Cushing's disease, hyperglycemia, acne, muscle weakness, osteoporosis, and cataracts, among other things.

Yet another such disease is colorectal cancer. The majority of colorectal cancers, regardless of etiology, are believed to arise from adenomatous polyps. These polyps protrude from the mucosa, and are visible endoscopically. Regular lower gastrointestinal screening and removal of polyps remains, by far, the best way to prevent colon cancer. Unfortunately, colon cancer still remains the second leading cause of cancer death in the U.S., primarily because of an unsatisfactory adherence to a regimented screening program. Certain hereditary syndromes (like Familial Polyposis) are characterized by the appearance of thousands of adenomatous polyps throughout the large bowel. If left surgically untreated, colorectal cancer will develop in almost all patients prior to age 40. To prevent colon cancer in these individuals, a total colectomy is usually required. There is currently no other hard and fast way to prevent colon polyps and thus colorectal cancer, although dietary factors, such as enhancing fiber and lowering saturated fat intake, might help. Non-steroidal anti-inflammatory drugs such as sulindac and celecoxib hold some promise. However, these nonsteroidal agents frequently produce adverse gastrointestinal side effects, renal failure, edema, and hypertension.

### SUMMARY OF THE INVENTION

The background art does not teach or suggest a biotherapeutic composition containing rapidly activatable bacteria in a dry form. The background art also does not teach or suggest such a composition for treatment of various intestinal disorders, including but not limited to, microbial infection, irritable bowel syndrome (IBS) and inflammatory bowel disease (IBD).

The present invention overcomes this deficiency of the background art by providing a biotherapeutic composition containing rapidly activatable bacteria in a dry form. The present invention also comprises a device for reconstituting and increasing the activated biomass, administering such a composition, and methods of treatment thereof. The present invention also comprises a method for preparing the biotherapeutic composition itself, as well as a method for preparing the bacteria for such a composition.

The biotherapeutic composition of the present invention includes, as a first element, bacteria in a dry form. By “dry form” it is meant that the bacteria are in a dried form, including but not limited to, a powder, a granulate, or a solid. By “dried” it is meant that the total moisture content of the bacteria is preferably less than about 10%, more preferably less than about 5% and most preferably less than about 1%. The bacteria may optionally be freeze dried, air dried or lyophilized, although any method for drying the bacteria may optionally be used.

The biotherapeutic composition of the present invention also includes, as a second element, a separate moist component for moistening the dry bacteria before administration to the subject. The moist component preferably includes a liquid medium, such as an aqueous medium for example. More preferably, the aqueous medium includes a solution, such as a sterile salt solution for example, although optionally the solution may include any substance suitable for administration to the subject. More preferably, the subject is a human, although optionally the subject may be a lower mammal. The moist component may alternatively comprise a semi-solid formulation, such asa pudding or yoghurt, or other formulation having such a consistency or texture. However, optionally and preferably, one or more taste or flavoring agents are included in the “dry” mixture with the probiotic bacteria itself.

The two elements are maintained in a separate state until the composition is to be administered to the subject. For example, the two elements of the biotherapeutic composition may optionally be stored in two separate compartments of a device. A non-limiting example of such a device is described below. The two elements are then mixed and administered to the subject, for example in a drink form.

According to optional but preferred embodiments of the present invention, the bacteria for the biotherapeutic composition have been selected according to at least one selection pressure. Optionally, the selection pressure may comprise at least one of temperature, time (stability when stored for a period of time), and osmotic pressure. The present invention also provides a method for preparing the biotherapeutic composition, comprising: selecting bacteria according to a selection pressure; and drying the bacteria. Optionally, as described in greater detail below, one or both of the second element (moist component) or the dried bacteria may be mixed with additional excipient(s). Non-limiting examples of such excipient(s) include flavoring agents, stabilizers, sugars or other energy sources, buffering agents and so forth.

The present invention also provides a method for treating a subject, comprising administering the biotherapeutic composition to the subject in need of treatment thereof, more preferably by providing the two elements of the composition in separate compartments of a device, and then mixing these two elements for administration to the subject. Preferably, the method is for treating a gastrointestinal disease or disorder for which treatment is desired or required, which may optionally and more preferably comprise a microbial infection, such as a bacterial infection, and/or IBD and/or IBS. The present invention is also useful for treatment of AAD (antibiotic associated diarrhea), as well as any form of acute diarrhea, for example caused by microbes (including but not limited to, enterotoxigenic E. coli, Salmonella, Proteus, Pseudomonas, Clostridium, Staphylococcus, Shigella flexneri and others), or by undetected pathogens; the syndrome of traveler's diarrhea; acute diarrhea in a hospital setting; as well as for treatment of the symptoms of diarrhea-associated IBS (Irritable Bowel Syndrome) whether mucous or inflammatory, and of diarrhea caused by radiation or chemotherapy.

The present invention is also useful for treatment of the various disease states related to the presence of “abnormal” or an “abnormal” distribution of microflora in the gastrointestinal tract; IBD (inflammatory bowel disease) whether mucous or inflammatory, spastic colon, mucous colitis, antibiotic-associated colitis, idiopathic or simple constipation, and chronic gastrointestinal infections with specific microorganisms such as Clostridium difficile, Campylobacter jejuni/coli etc. and Candida; and chronic diarrhea due to disturbances of the digestive tract microbe balance caused by antibiotics, radiation therapy or chemotherapy, intestinal infection, digestive tract surgery, immunodeficiency, the effects of an unfavorable ecological situation, including higher radiation and age changes; microscopic or lymphocytic colitis, collagenous colitis, colon polyps and familial polyp syndromes (e.g., familial polyposis syndrome, Gardner's Syndrome).

According to other preferred embodiments of the present invention, the composition and method are optionally useful for treating food intoxication, dyspeptic symptoms or episodes of acute diarrhea, or diarrhea caused by undetected pathogens or unknown etiology. The present invention is also optionally useful for treating diseases and disorders of the digestive tract caused or maintained by disturbance of the microbial balance of the intestinal microflora, and/or by a bacterial overgrowth in the small intestine. The present invention is also optionally useful for preventing or decreasing a level of disturbance microbial balance of the digestive tract microflora resulting from antibiotic therapy, radiotherapy or chemotherapy, diseases or disorders of the digestive tract, including digestive tract surgery.

According to yet other preferred embodiments of the present invention, the composition and method are optionally useful for preventing or treating disturbances in microbial balance of the digestive tract microflora resulting from diseases outside of the digestive tract, certain dietary and environmental factors. The present invention is also useful for improving or normalizing the physiological activity of the gastrointestinal tract in elderly and in the compromised patients.

Hence, according to one aspect of the present invention there is provided a method of treating an inflammatory bowel disease/irritable bowel syndrome (IBD or IBS, and others) in a subject in need thereof. The method comprises orally administering to the subject a therapeutically effective amount of a probiotic Escherichia coli strain in a mixed formulation, containing the two elements of the composition in a mixture that is prepared before administration. The therapeutically effective amount preferably ranges between about 10⁶ and about 10¹² viable bacteria per administration, ranging from 1 to 10, preferably about 2-4 administrations per day.

According to a further aspect of the present invention, there is provided method of treatment for microbial infection, the method comprising orally administering to the subject a therapeutically effective amount of a probiotic strain in a mixed liquid or semi-solid formulation, preferably an Escherichia coli strain, in which the two elements are kept separated and are then mixed before administration, preferably in a device featuring two separate compartments for storage. More preferably, the elements are mixable in the device and may then be administered to the subject, optionally from the device itself.

The table below shows suggested doses of the composition according to the present invention for treatment of various diseases and disorders, and is meant for illustrative purposes only, without wishing to be limiting in any way. The doses are given according to a measurement of the biotherapeutic composition in its mixed form.

Table of Exemplary Diseases/Disorders and Suggested Dosing Regimens

| | |
|---|---|
| Disease/Disorder | Suggested Doses |

1. Diarrhea

| | |
|---|---|
| Bacterial (Salmonella, Shigella, Staphylococci, E. coli, | 1-3 tablespoons every 3-4 hours until diarrhea is |
| Pathogenic serotypes, Klebsiella etc) | discontinued or the rate decreases; after which |
| | 1 tablespoon 3 times per day for 7-10 days |
| Diarrhea associated with antibiotics | 1 tablespoon 3 times per day |
| Traveler's diarrhea | 1-3 tablespoons every 3-4 hours |
| Occasions of acute diarrhea of unknown etiology | 1-3 tablespoons every 3-4 hours |
| Diarrhea after intestinal surgery or after removal of gall | 1 tablespoon 2-3 times per day |
| bladder | |
| Associated with diabetes | 1 tablespoon 3-4 times per day for 3-4 months |
| After exposure to radiation and chemotherapy | 1 tablespoon 3 times per day |
| Age-related | 1 tablespoon 3 times per day for 3-4 months |
| Viral | 1 tablespoon 3 times per day |
| Parasite related | Preferably as a supplemental treatment, 1 tablespoon 3 |
| | times per day |

2. Constipation

| | |
|---|---|
| Age related | 1 teaspoon 3 times per day |
| After chemotherapy | 1 tablespoon 3 times per day |
| Associated with diabetes | 1 teaspoon 3 times per day |
| 3. Irritable intestinal syndrome | 1 tablespoon 2-3 times per day for 3-4 months |
| 4. pathology (abnormality) in intestinal micro-ecologic | 1 tablespoon 3 times per day for 3-4 months |
| balance (dysbacteriosis, including candidosis | |
| accompanied by discomfort, excessive flatulence and | |
| periodic pains in the stomach, belching, bad breath, | |
| symptoms indicating deficiency of vitamins B12, B1, | |
| B2, and so forth | |

The present invention is also useful for improving or normalizing the immune system in subjects suffering from an immune system disorder, including disorders as side effect caused by therapy of other diseases, as well as being useful for treating domestic animals.

According to still further features in the described preferred embodiments, the probiotic non-pathogenic lactose-positive strain, such as the Escherichia coli strain M-17, is provided alone, or optionally with one or more E. coli strains and/or other bacterial strains.

According to still further features in the described preferred embodiments, the mixed formulation (containing the mixture of the two elements of the composition) comprises between about 10⁶ and about 10¹² CFU per ml of the probiotic Escherichia coli strain, more preferably from about 10⁷ to about 10⁸ CFU per ml of the probiotic Escherichia coli strain.

According to an additional aspect of the present invention there is provided a dispenser for dispensing a biotherapeutic composition comprising rapidly activatable bacteria in a dry form, a carrier for containing at least one substance for causing rapid activation of the bacteria and a moist component, wherein the dispenser comprises a first compartment for holding the moist component, a second compartment for holding the bacteria in a dry form, and a separator for separating the first and second compartments, such that when the separator is at least partially removed, the moist component and the dried bacteria are permitted to mix to form a mixture, thereby forming the biotherapeutic composition.

According to further features in the described preferred embodiments, the separator may be partially removable by one of piercing, detaching, removing and separating.

According to further features in the described preferred embodiments, the dispenser may optionally further comprise a rod having a first end extending out of the body of the dispenser and a second end positioned adjacent to the separator, the rod being depressible within the body, such that manipulation of the rod causes the second end to push against the separator, wherein the separator is pierced or removed. The rod may optionally be, for example, a spring loaded plunger, which may further optionally be provided with a handle attached to the first end. Manipulation of the rod may comprise, for example, at least one of pushing and twisting.

According to further features in the described preferred embodiments, one of the compartments may optionally comprise a bottle.

According to still further features in the described preferred embodiments, the dispenser may further comprise a nozzle in communication with the first compartment. A removable cover may optionally be further provided for sealing the nozzle.

According to further features in the described preferred embodiments, the separator may be positioned over an aperture between the first and second compartments, a first part of the separator being connected to a first side of the aperture by a break-away score for being broken, and a second part of the separator being connected to a second side of the aperture by a hinge score, such that when pressure is exerted upon the break-away score, the break-away score becomes broken, and the second part of the separator swings on the hinge score, thereby opening the aperture between the first and second compartments.

According to further features in the described preferred embodiments, the separator may be a membrane, which is impermeable to the moist component. Optionally, the membrane comprises a portion of an inner cap sealing an open upper portion of a lower of the first and second compartments.

The membrane may optionally be removable by piercing, detaching, removing and separating. Preferably, the membrane is removable by piercing. The dispenser may optionally further comprise a piercing member positioned over said membrane, which may optionally comprise an integral part of the upper of the first and second compartments. Also optionally, the dispenser -may further comprise a depressible member, such as a plunger, positioned over the piercing member, such that when the depressible member is depressed, the piercing device is caused to pierce the membrane. The depressible member optionally forms a portion of an outer cap positioned over and engaged by the inner cap. The dispenser may optionally further comprise a removable safety device, such as a pull-tab, which may be positioned beneath a lower edge of the outer cap, for preventing depression of the depressible member.

The present invention successfully addresses the shortcomings of the presently known configurations by providing a method and a biotherapeutic pharmaceutical composition for treating bacterial infections and/or inflammatory bowel disease/irritable bowel syndrome (IBD or IBS, or others) with a probiotic E. Coli strain. Such treatment is highly advantageous as compared with the present methods of treating such diseases or disorders as described above, or other diseases or disorders, as it is efficacious, safe, non-invasive and free of side effects.

An advantage of the present invention is that the probiotic action of the bacteria commences immediately upon reaching the gastrointestinal tract, because of the mixing of the moist component and the dry bacteria before administration to the subject.

A further advantage of the present invention is that the preparation may be stored for long periods of time without significant loss of bacterial viability.

The present invention also has the advantage that the wide spectrum of efficacy of the biotherapeutic composition enables intestinal infections to be treated effectively without first identifying the pathogen and defining its sensitivity to antibacterial preparations.

### BRIEF DESCRIPTION OF THE DRAWINGS

The invention is herein described, by way of example only, with reference to the accompanying drawings:

FIG. 1 shows an illustrative embodiment of the device according to the present invention;

FIG. 2 shows a cross-section of the device of FIG. 1 in a storage and/or transport format, with at least two separate compartments;

FIG. 3 shows a cross-section of the device of FIG. 1 after contents of the at least two separate compartments have been allowed to mix;

FIG. 4 shows mixing of the contents of the at least two separate compartments of the device of FIG. 1;

FIGS. 5A and 5B show two exemplary embodiments of the nozzle for administration of the resultant mixture for the device of FIG. 1;

FIG. 6 shows a cross-section of another exemplary embodiment for the device according to the present invention; and

FIG. 7 shows a cross-section of a further exemplary embodiment of the device according to the present invention.

### DETAILED DESCRIPTION OF THE INVENTION

The present invention is of a biotherapeutic composition containing rapidly activatable bacteria in a dry form. The present invention also comprises a device for reconstituting and increasing activated biomass, administering such a composition, and methods of treatment thereof. The present invention also comprises a method for preparing the biotherapeutic composition itself, as well as a method for preparing the bacteria for such a composition.

The biotherapeutic composition of the present invention includes, as a first element, bacteria in a dry form. By “dry form” it is meant that the bacteria are in a dried form, including but not limited to, a powder, a granulate, or a solid. By “dried” it is meant that the total moisture content of the bacteria is preferably less than about 10%, more preferably less than about 5% and most preferably less than about 1%. The bacteria may optionally be freeze-dried, air dried or lyophilized, although any method for drying the bacteria may optionally be used.

The biotherapeutic composition of the present invention also includes, as a second element, a separate moist component for moistening the dry bacteria before administration to the subject. The moist component preferably includes a liquid medium, such as an aqueous medium for example. More preferably, the aqueous medium includes a sterile solution, such as a sterile salt solution for example, although-optionally the sterile solution may include any substance suitable for administration to the subject. The moistened combination may optionally comprise suspensions or solutions in water or non-aqueous media. More preferably, the subject is a human, although optionally the subject may be a lower mammal. The moist component may alternatively comprise a semi-solid formulation, such as a pudding or yoghurt, or other formulation having such a consistency or texture. The moist component also preferably comprises at least one other ingredient for increasing the palatability of the composition, for example with regard to taste, smell or texture, or a combination thereof.

The two elements are maintained in a separate state until the composition is to be administered to the subject. For example, the two elements of the biotherapeutic composition may optionally be stored in two separate compartments of a device. A non-limiting example of such a device is described below. The two elements are then mixed and administered to the subject, for example in a drink form (solution or suspension) and/or a swallowable or otherwise ingestible semi-solid formulation, such as a gel, a pudding, a thickened paste or other thickened composition, or any other semi-solid formulation. Alternatively, the two elements are mixed, and the biotherapeutic component is first permitted to reactivate and multiply within the device prior to administration to the subject.

Ease of administration is only one of the many advantages of combining the two elements before administration to the subject. A liquid or semi-solid composition may be administered to a subject with relative ease, even to a child, an elderly subject, and/or a handicapped subject, or any other subject who may experience difficulty in swallowing a pill or other solid dosage form. However, the mixed composition preferably includes at least one ingredient for enabling the bacteria to become more rapidly activated, more preferably before administration of the composition to the subject. Therefore, the composition of the present invention preferably enables the bacteria to be stored in a dry form, yet to be “jump-started” for rapid activation, optionally before or after administration of the composition to the subject.

According to optional but preferred embodiments of the present invention, the bacteria for the biotherapeutic composition have been selected according to at least one selection pressure. By “section pressure” is meant an unfavorable condition to which bacterial cells are subjected in order to select those cells which remain viable under such conditions. Optionally, the selection pressure may comprise at least one of temperature, time (stability when stored for a period of time), and osmotic pressure, as is detailed hereinbelow.

The present invention also provides a method for preparing the biotherapeutic composition, comprising: selecting bacteria according to a selection pressure; and drying the bacteria. Thus, bacterial cells are initially selected by application of selection pressure factors, in order to select those cells which remain viable upon being subjected to conditions unfavorable to metabolism. These selection pressure factors may optionally and preferably include at least one of time (stability when stored for a period of time), temperature, and osmotic pressure conditions. Hence, bacteria having maximum survival ability are selected.

Temperature selection conditions may optionally and preferably comprise subjecting the cells to temperatures which exceed the optimum range for active vital cell metabolism, preferably to temperatures of about 40° C. for a period of between 4 and 5 days.

Preferably, cells may be selected by subiecting to temperatures which are below the optimum temperature range for active vital cell metabolism, preferably temperatures of between about 2° C. and about 15° C. for a period of between 1-12 months, and more preferably, for between 3 and 12 months.

According to the method of the present invention, selected bacteria are preferably used to inoculate a growth medium, for production of a biomass containing selected, viable non-pathogenic bacteria, optionally and preferably comprising between about 10⁷ and about 10⁸ colony forming units (CFUs) of the selected probiotic Escherichia coli per ml. The suspension medium is preferably essentially free from growth medium.

The suspension medium optionally and preferably promotes autolysis under conditions which prevent production of biodegrading components of bacterial cells. Autolysis may optionally be increased by application of mechanical actions and/or through the composition of the environment. For example, autolysis may be induced by provision of an osmotic imbalance between the osmotic pressure inside the bacterial cell and that of the suspension medium. For example, autolysis may be induced by use of a suitable suspension medium having low osmotic pressure, most preferably from about 0.3% to about 0.6% sodium chloride solution.

Alternatively, autolysis may be induced through changes to the density of the bacterial suspension, for example by causing the density to preferably be from about 10¹¹ to about 10¹² number of bacteria per ml (CFU; it should be noted that these two terms are used interchangeably in the application).

Also alternatively, another method may be used, to prevent the production of biodegradation components of the bacteria. Examples of such a method include but are not limited to ultrasound or other methods, for example.

Optionally and preferably, one or both of the second element (moist component) or the dried bacteria may be mixed with additional excipient(s).

According to an optional but preferred embodiment of the present invention, one or more excipients are mixed with the dry bacteria. Such excipients may optionally be mixed after the bacteria have been dried, for example by mixing the excipient(s) and the bacteria in a powder form.

Alternatively or additionally, one or more excipient(s) may optionally be added to the bacteria in a liquid form, after which the combination is dried. For example, U.S. Pat. No. 6,569,424, hereby incorporated by reference as if fully set forth herein, describes combining the bacteria with a carbohydrate enriched media, whereby the bacteria and media are combined and allowed to ferment until a desired number of total organisms per dose is achieved. The bacterial component of the biotherapeutic composition may then optionally and preferably be concentrated and lyophilized. The carbohydrate-enriched media includes any such media as is common in the art. One embodiment of the present invention includes a carbohydrate enriched media that is a dairy product. Any dairy product may be appropriate, but milk is particularly useful as the media.

Excipients suitable for use in the present invention include, for example, flavoring agents, stabilizers, sugars or other energy sources, buffering agents, thickeners, diluents, dispersing aids, emulsifiers or binders and so forth.

Stabilizers/emulsifiers are well known to the art, and are used in various food products to enhance and maintain the desirable characteristics of the product, e.g. body and texture, viscosity/consistency, appearance and mouth-feel. Examples of such stabilizers/emulsifiers include but are not limited to: natural gums; modified natural or semi-synthetic gums; and synthetic gums. Gelatin and modified gelatin may also optionally be used. Non-limiting examples of stabilizers/emulsifiers suitable for use in the present invention may be found in Tamine and Robertson, Yoghurt Science and Technology 1985, Pergamon Press, also hereby incorporated as if fully set forth herein.

The stabilizers/emulsifiers can be used at a concentration of about 0.1 to about 25 weight percentages. It will be appreciated that the concentration can vary depending on the type of product, the amount of starch and/or dietary fiber (or any other carrier ingredient that is used), and the probiotic microorganisms.

The biotherapeutic composition according to the present invention optionally and preferably includes a carrier, which acts as a growth or maintenance medium for microorganisms, at least before being placed in contact with the gastrointestinal tract, but also optionally after administration to the gastrointestinal tract. Such a carrier may optionally be included with the dry bacteria and/or with the moist component, or both. Alternatively, multiple such elements may be provided, with the dry bacteria as a first element, the carrier as a second element, and the moist component as a third element. Other such elements may also optionally be provided. Preferably, such elements are packaged in separate compartments of a single device, and are more preferably mixable within the device.

Examples of suitable ingredient(s) for the carrier include but are not limited to, trehalose, malto-dextrin, rice flour, micro-crystalline cellulose (MCC), magnesium sterate, inositol, FOS, glucooligosaccharides (GOS), dextrose, sucrose, talc, and the like. Additional carriers suitable for use in the present invention will suggest themselves to one skilled in the art.

Either the carrier or the stabilizer may optionally further comprise a vegetable gum, such as an alginate, preferably calcium alginate, which forms a gel for encapsulation of the bacteria, thereby increasing survival of the bacteria during storage and gastrointestinal transit.

If the carrier includes evaporated oils that produce a tendency for the composition to cake (adherence of the component spores, salts, powders and oils), it is preferred to include dry fillers which distribute the components and prevent caking. Exemplary anti-caking agents include MCC, talc, diatomaceous earth, amorphous silica and the like, typically added in an amount of from about 1 weight percentage to about 95 weight percentages.

The carrier may also optionally comprise a rehydration formulation for rehydration of the bacteria that includes glucose, potassium citrate, sodium chloride and/or sodium citrate, as a non-limiting example of a suitable rehydration formulation.

Well known thickening agents may be added to the composition, such as corn starch, guar gum, xanthan gum, calcium alginate, and the like. Preservatives may also be included in the carrier, including methylparaben, propylparaben, benzyl alcohol and ethylene diamine tetraacetate salts. Well-known flavorings and/or colorants may also be included in the carrier. The composition may also include a plasticizer such as glycerol or polyethylene glycol.

The moist component may optionally include an aqueous or oleaginous base, such as, for example, white petrolatum, isopropyl myristate, lanolin or lanolin alcohols, mineral oil, fragrant or essential oil, nasturtium extract oil, sorbitan mono-oleate, propylene glycol, cetylstearyl alcohol (together or in various combinations), hydroxypropyl cellulose (MW=100,000 to 1,000,000), detergents (e.g., polyoxyl stearate or sodium lauryl sulfate). Alternatively or additionally, one or more of the base ingredients in dry form may optionally be mixed with the dried bacteria, and/or may be present as a separate carrier element. When mixed with the dried bacteria (and if necessary the separate carrier element), the combination of these two elements optionally and preferably form a lotion, gel, cream or semi-solid composition.

Other suitable moist components comprise water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-I 5-stearyl ether and water. For example, emulsions containing water, glycerol stearate, glycerin, mineral oil, synthetic spermaceti, cetyl alcohol, butylparaben, propylparaben and methylparaben are commercially available.

The present invention also provides a method for treating a subject, comprising administering the biothetrapeutic composition to the subject in need of treatment thereof, more preferably by providing the two elements of the composition in separate compartments of a device, and then mixing these two elements for administration to the subject. Preferably, the method is for treating a gastrointestinal disease or disorder for which treatment is desired or required, which may optionally and more preferably comprise a microbial infection, such as a bacterial infection, and/or IBD and/or IBS. The present invention is also useful for treatment of AAD (antibiotic associated diarrhea), as well as any form of acute diarrhea, for example caused by microbes (including but not limited to, enterotoxigenic E. coli, Salmonella, Proteus, Pseudomonas, Clostridium, Staphylococcus, Shigella flexneri and others), or by undetected pathogens; the syndrome of traveler's diarrhea; acute diarrhea in a hospital setting; as well as for treatment of the symptoms of diarrhea-associated IBS (Irritable Bowel Syndrome) whether mucous or inflammatory, and of diarrhea caused by radiation or chemotherapy.

The present invention is also useful for treatment of the various disease states related to the presence of “abnormal” or an “abnormal” distribution of microflora in the gastrointestinal tract; IBD (inflammatory bowel disease), whether mucous or inflammatory, spastic colon, mucous colitis, antibiotic-associated colitis, idiopathic or simple constipation, and chronic gastrointestinal infections with specific microorganisms such as Clostridium difficile, Campylobacter jejuni/coli etc. and Candida; and chronic diarrhea due to disturbances of the digestive tract microbe balance caused by antibiotics, radiation therapy or chemotherapy, intestinal infection, digestive tract surgery, immunodeficiency, or the effects of an unfavorable ecological situation, including higher radiation and age changes.

According to other preferred embodiments of the present invention, the composition and method are optionally useful for treating food intoxication, dyspeptic symptoms or episodes of acute diarrhea, or diarrhea caused by undetected pathogens or unknown etiology. The present invention is also optionally useful for treating diseases and disorders of the digestive tract caused or maintained by disturbance of the microbial balance of the intestinal microflora, and/or by a bacterial overgrowth in the small intestine. The present invention is also optionally useful for preventing or decreasing a level of disturbance of microbial balance of the digestive tract microflora resulting from antibiotic therapy, raidiotherapy or chemotherapy, diseases or disorders of the digestive tract, including digestive tract surgery.

According to yet other preferred embodiments of the present invention, the composition and method are optionally useful for preventing or treating disturbances in microbial balance of the digestive tract microflora resulting from diseases outside of the digestive tract, such as certain dietary and environmental factors. The present invention is also useful for improving or normalizing the physiological activity of the gastrointestinal tract in elderly and/or compromised patients.

Hence, according to one aspect of the present invention there is provided a method of treating an inflammatory bowel disease/irritable bowel syndrome (IBD or IBS, and others) in a subject in need thereof. The method preferably comprises orally administering to the subject a therapeutically effective amount of a probiotic Escherichia coli strain in a mixed formulation, containing the at least two elements of the composition in a mixture that is prepared before administration. The therapeutically effective amount preferably ranges between about 10⁶ and about 10¹² viable bacteria per administration, ranging from 1 to 10, preferably about 2-4 administrations per day.

According to a further aspect of the present invention, there is provided a method of treatment for microbial infection, the method comprising orally administering to the subject a therapeutically effective amount of a probiotic strain in a mixed liquid or semi-solid formulation, preferably an Escherichia coli strain, in which the two elements are kept separated and are then mixed before administration, preferably in a device featuring two separate compartments for storage. More preferably, the elements are mixable in the device and may then be administered to the subject, optionally from the device itself.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein, the term “treating” includes abrogating, substantially inhibiting, slowing or reversing the progression of a disease, substantially ameliorating clinical symptoms of a disease or substantially preventing the appearance of clinical symptoms of a disease.

The term “preventing” refers to barring a subject from acquiring a disorder or disease in the first place.

As used herein, the phrase “inflammatory bowel disease (IBD)” refers to a disorder or disease characterized by inflammatory activity in the GI tract, and may include mucosal forms of IBD. Examples of IBDs that are treatable by the probiotic strains of the invention include, without limitation, Crohn's disease (both distal and proximal), ulcerative colitis, indeterminate colitis, microscopic colitis, collagenous colitis, idiopathic inflammation of the small and/or proximal intestine and IBD-related diarrhea.

The term “administering”, as used herein, refers to a method for bringing the probiotic E. coli strain(s) or other strain(s) into an area or a site in the GI tract that is affected by the disease or disorder.

The term “therapeutically effective amount” refers to that amount of a probiotic E. coli strain or other strain being administered, which will relieve to at least some extent one or more of the symptoms of the disease or disorder being treated.

Hereinafter, the term “subject” refers to the human or lower animal to which the therapeutic agent is administered.

Dosing is dependent on the severity of the symptoms and on the responsiveness of the subject to the therapeutic agent. Persons of ordinary skill in the art can easily determine optimum dosages, dosing methodologies and repetition rates.

A therapeutically effective amount, according to the method of the present invention, preferably ranges between about 10⁶ and about 10¹² viable bacteria per administration, more preferably between about 10⁷ and about 10¹⁰ viable bacteria per administration, more preferably between about 10⁸ and about 10¹⁰ viable bacteria per administration and most preferably it is between about 5×10⁹ and about 2×10¹⁰ viable bacteria per administration.

The number of administrations according to the present invention preferably ranges between 1 and 10 administrations per day, more preferably between 1 and 5 administrations per day and most preferably between 2 and 4 administrations per day. The overall amount of viable bacteria that is administered daily preferably ranges between about 10⁹ and about 10¹¹ viable bacteria per day, although it may optionally range between about 10⁶ and about 10¹² viable bacteria per day.

The probiotic strain of the present invention is preferably initially formulated as a dry composition, but administered as a liquid or semi-solid formulation, as is described in detail hereinbelow and is further exemplified in the Examples section that follows.

According to an optional but preferred embodiment of the present invention, the mixture of the dried bacteria and the moist component (and if necessary the separate carrier) is allowed to stand before administration to the subject. Optionally the mixture is allowed to stand at least for a predetermined period of time. Alternatively, the mixture is allowed to stand at least until a particular endpoint is reached, such as a change in pH of the mixture (optionally measured through a change in color of a pH sensitive substance), or an increase in optical density, which indicates that the bacteria have become at least somewhat activated. Such pre-activation causes the bacteria of the biotherapeutic composition of the present invention, for example, to be therapeutically active immediately or at least shortly following oral administration, as little or no biomass generation in the gut is preferably required.

The mixture of the probiotic strain, according to the present invention, once prepared, may optionally include salt in an isotonic amount and can further comprise other ingredients, as further detailed hereinbelow. Preferably, the resultant mixture has a pH which is favorable for maintaining viability.

The prepared mixture of the probiotic strain, according to the present invention, typically comprises between about 10⁵ and about 10¹² CFU (colony forming units) of the probiotic Escherichia coli strain, per ml (or other strain). Preferably, the mixture comprises between about 10⁶ and about 10¹⁰ CFU per ml, more preferably between about 10⁷ and about 108 CFU per ml.

Non-pathogenic lactose-positive E. coli, such as strain M17, strain Nissle and other strains are preferred examples of bacterial strains for use with the present invention, as they comprise the main group of healthy aerobic microflora in the intestine of humans and animals, providing microbiological balance and playing an important role in alimentation and immunity.

This strain of bacteria belongs to the same phylogenetic group as the majority of intestinal pathogens responsible for causing diarrhea; therefore their survival conditions are largely similar, resulting in a high level of competitive exclusion between the strains. This competitive effect includes production of antimicrobial substances during growth of probiotic bacteria, competition for nutrients and growth factors, synergistic nutrient utilization, and competition for receptor sites.

The antagonistic effect of the biotherapeutic composition of the present invention on bacterial pathogens was found to be considerably higher than that of probiotic bacteria from standard freeze-dried preparations. It should be noted that by “antagonistic”, it is meant the ability of a particular bacterial strain to antagonize growth of other bacteria or other microorganisms.

It is known that the action of gastric juice, largely comprising hydrochloric acid, causes death of many bacteria. Bacteria in dried form are weaker than those contained in liquid medium, and are therefore more susceptible to the effects of gastric juice. The bacteria contained in the biotherapeutic composition of the present invention, after preparation of the mixture, are therefore more stable upon passage through the stomach than those in standard freeze-dried preparations. Typical probiotic bacteria, such as Lactobacillus sp. and Bifidobacterium sp. enter the colon before beginning to multiply and exert their antagonistic properties. However, the site of primary action for the majority of intestinal pathogens is not the colon but the upper part of the gastro-intestinal tract. Known probiotic preparations do not enable delivery of a competitive concentration of live bacteria to the upper portions of the intestine, and are therefore practically ineffective in eliminating acute bacterial diarrhea and conditions caused by disturbance of the micro-ecological balance in upper sections of the intestine.

In preparation of the liquid biotherapeutic composition of the present invention, the E. coli bacterial cells (or other bacterial cells) having the highest antagonistic activity and the most persistent bacterial cells under storage for long periods of time, preferably up to about 12 months, are more preferably first selected from lactose-positive non-pathogenic E. coli species having beneficial probiotic properties.

E. coli cells or other bacteria for use in the biotherapeutic composition of the present invention are optionally and preferably selected by exerting selection pressure on the cells such that only selected cells emain viable. Application of selection pressure may be achieved by use of time pressure (stability over time), such that cells having long-term survival ability are selected; application of osmotic pressure; decrease of basal metabolism; or increase in temperature. Temperature selection optionally and preferably comprises subjecting the cells to temperatures of about 40° C. for at least 4 days, and/or to higher temperatures for a shorter period of time. By these means, only cells having high survival abilities are selected from the initial culture.

The selected bacterial cells were used for inoculation of a growth medium. A suitable growth medium preferably includes all of the necessary nutrients, growth factors etc as are known in the background art, such as described for example in “Manual of Methods for General Bacteriology”, P. Gerhardt ed., American Society for Microbiology, Washington, DC, USA, 1981.

It is known that osmotic pressure inside cells of Gram-negative bacteria, particularly E. coli, may reach up to about 15 atmospheres in the log phase of growth, and from 2 to about 3 atmospheres in the stationary phase of growth. In a preferred embodiment of the method of the present invention, a suspension medium having low osmotic pressure, preferably below 1 atmosphere, more preferably from about 0.3 to about 0.4 atmospheres, is used. Osmotic imbalance and high bacterial density during the first preparation stage of the strain for the biotherapeutic composition of the present invention create conditions for autolysis of the weakest and smallest stable bacterial cells in the log phase. These lysed cells provide an accumulation of cellular components from bacteria in the suspension medium, which provide nutritional requirements of remaining cells. Using this procedure, cell concentrations of from 10¹¹ to about 10¹² bacteria per ml (CFU) were obtained, although again cell concentrations may optionally be present in a broader range.

The biotherapeutic. composition of the present invention may be used in treatment of humans and of animals.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The formulation, preparation, device for administration and use of the biotherapeutic composition of the present invention are illustrated with reference to the following non-limiting examples.

### Example 1

### Process for the Preparation of the Bacteria

The selected bacteria are first prepared for growth to form the biomass in the form of concentrates ranging from 10¹¹-10¹² CFU per ml in 0.3%-0.6% NaCl solution, to produce the autolysate.

Liquid Medium:

For bacterial biomass preparation a standard fermentation vessel with aeration can be used. Nutrients necessary for bacterial growth are added in two stages: in a first stage, as part of the original batching medium, and in a second stage, following nutrient depletion in the production reactor, as a continuous supplemental feeding solution.

In a typical fermentation process, a medium may consist of a suitable nitrogen source, glucose, sodium chloride, and a combination of disodium phosphate and monopotassium phosphate sufficient to provide a neutral or slightly basic pH (7.2±0.2).

An exemplary medium includes phosphate salts such as, for example, sodium and potassium phosphates; magnesium sulfate; halide salts such as, for example, sodium, ammonium and calcium chlorides; trace minerals and nicotinic acid, with glucose as an energy source.

Additional nutrients are automatically supplied into the nutrient medium during the process of bacterial growth.

Additional glucose should be continuously added following the growth of the culture in such way that the glucose concentration in the fermentation broth is kept at a constant-level.

Additional aeration (0.5 vvm) is performed during the entire period of bacterial growth.

The pH of the fermentation broth may be kept neutral by the continuous addition of 4N NH₄OH.

The broth is incubated at temperatures of from about 32 to about 36° C. until the stationary phase of the growth cycle is reached.

After 16-18 hours, the cells are harvested by centrifugation or ultrafiltration, up to a level at which residual quantities of total nitrogen are not more than 0.3%, and preferably not more than 0.03 % for cell concentration of 10⁷-10⁸ microbial cells per ml of suspension, resuspended in saline and re-precipitated.

A 10¹¹-10¹² suspension of the bacteria is prepared in 0.4 %-0.6 % NaCl or any other suitable chemically defined salt solution, such as M9 (Na₂HPO₄ 6 g/L, KH₂PO₄3 g/L, NaCl 0.5 g/L and NH₄Cl 1 g/L) cooled to 4-8° C. and, stored under refrigerated conditions. It should be noted that the concentration of bacteria for this stage (and/or for administration to the subject) may optionally range from about 10⁶ to about 10¹² bacteria per ml.

Solid Culture Media:

Non-pathogenic E. coli were grown on a solid culture medium, using a composition of nutrients providing maximum accumulation of bacterial biomass according to the present invention.

The medium optionally and preferably includes a nitrogen source, dextrose, sodium chloride, and agar. The final pH of the medium is preferably about 7.

An exemplary composition of the medium is as follows:

Formula (in g/l)

| | |
|---|---|
| Soy peptone | 10.0 |
| Yeast extract | 18.0 |
| Dextrose | 2.5 |
| Sodium chloride | 4.0 |
| Agar | 12.0 |

Final pH 7.0 (0.2 approx.)

Prepared medium is poured into corresponding matrices with layer thickness of 5-7 millimeters. After cooling, the culture medium is seeded with bacterial culture E. coli M-17.

Matrices are placed in an incubator and incubated under aerobic conditions at the optimum temperature (34-38° C.) for about 24-28 hours. This procedure yielded 10¹⁰-10¹¹ cells/ml of the culture medium.

After this period, the isolated pure culture should be removed from plates by “Dry method”, in which the bacteria are removed with a tool such as a spatula, without introducing a liquid (or at least substantially quantities of a liquid) to the plates. For this purpose special adjustments for biomass collection have been used.

A 10¹¹-10¹² CFU suspension of the bacteria is prepared in 0.4%-0.6% NaCl solution. The suspension is optionally and preferably stored under refrigerated conditions for the time and storage pressure embodiment of the present invention.

### Example 2

### Preparation of the Biotherapeutic Composition—Exemplary Method

The composition according to the present invention may optionally be prepared according to the following exemplary method. Probiotic E. coli (10⁸-10⁹ cells), optionally from a seed stock, are inoculated into liquid or solid culture medium components using standard microbial fermentation techniques. Growth conditions preferably include continuous aeration, maintenance of neutral pH and supplementation with glucose. This organism has preferably not been genetically engineered in any way, but rather has been isolated from microflora obtained from a normal human gastrointestinal tract.

Manufacturing is optionally and preferably controlled with respect to the following critical control points:

Precautions to be taken receiving and handling cultures;

Control procedures to assure appropriate culture conditions;

Maintenance of sterility;

Control procedures to assure correct levels of probiotic bacteria in finished product.

Optionally and preferably the seed stock itself may be prepared as follows. One frozen vial of E. coli M-17 strain is removed from storage at −80° C., thawed at room temperature, and then transferred aseptically into a sterile baffled shake flask containing sterilized Tryptic Soy broth (Difco). After 15-20 hour's growth, the culture is examined microscopically and streaked onto a Bacto m Endo Agar LES plates to check for purity.

Alternatively, the seed stock may be produced by removing one frozen vial of E. coli M17 from storage at −80° C., placing it in a dry heat block at 37° C. to thaw, and then transferring aseptically into a sterile baffled shake flask containing sterilized M9 medium. After 8-16 hours' growth, the culture is streaked onto replicate Trypticase soy agar (TSA) and Levine Eosin Methylene Blue (EMB) agar plates to check for purity. Seed culture may be expanded in a stepwise fashion (e.g. from 10 mL to 50 mL to 1000 mL prior to introduction into the bioreactor.

Reactor Preparation

Each reactor is batched and sterilized with the medium in place. The glucose solution is sterilized separately and added to a concentration of 2.5 g/L before culture inoculation.

Reactor Inoculation:

The seed culture is aseptically transferred to the bioreactor, and the culture grown under established conditions of temperature, pH, agitation and dissolved oxygen. Glucose solution feeding at a rate of 10-12 mL/hour is started when the initial glucose concentration reaches 0 g/L. The culture is allowed to grow for ≦24 hours or until the OD₆₅₀ reaches a maximum of 20. The reactor is then cooled to below 10° C. for harvest.

Microfiltration:

Bioreactor contents are harvested by concentration using a 0.2 μm pore size tangential flow microfiltration unit. Concentrate is diafiltered with 5 volumes of sterile saline and then placed into sterile bottles for storage at 4-6° C. The sample is examined microscopically and streaked onto a Bacto m Endo Agar LES plate to check for purity and enumerated by plating onto Tryptic Soy agar plates.

As an alternative to microfiltration, the bioreactor contents may be harvested by batch or continuous centrifugation and repeated washing with sterile medium or salt solution and then placed into sterile bottles.

### Example 3

### Illustrative Devices for Administration

This Example describes a number of different non-limiting illustrative device embodiments for storage and administration of the biotherapeutic composition according to the present invention.

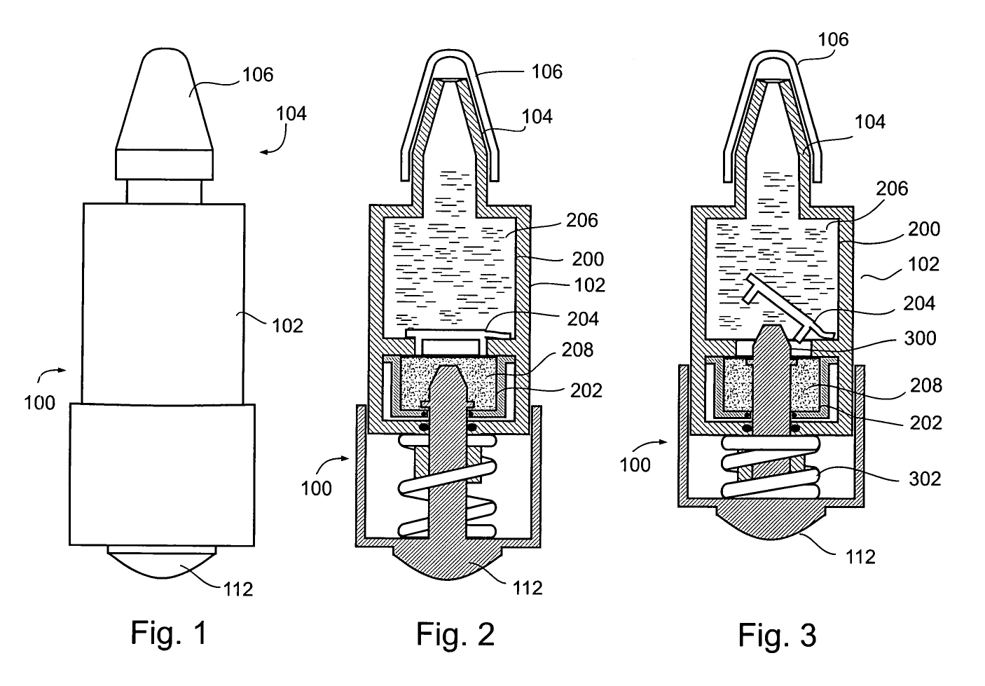

As shown with regard to FIG. 1, an exemplary device 100 according to the present invention features a body 102 for containing a plurality of compartments (not shown, see FIG. 2). Body 102 is preferably in communication with a nozzle 104 for administration of the mixture to the subject. Nozzle 104 is preferably covered with a cover 106 which may be removed for administration to be performed.

Body 102 is preferably divided into a plurality of portions, whose function is described in greater detail below with regard to FIGS. 2 and 3. Device 100 is also preferably provided with a handle 112, whose function is also described in greater detail below with regard to FIGS. 2 and 3.

FIGS. 2 and 3 show cross-sections of device 100. In FIG. 2, device 100 is shown with two compartments 200 and 202, separated by a separator 204. Compartment 200 optionally and preferably contains a moist component 206, while compartment 202 optionally and preferably contains dried bacteria 208, although these positions could be reversed. Optionally a separate carrier may be present in a separate compartment (not shown); alternatively, the carrier may optionally be mixed with the dried bacteria and/or the moist component.

Device 100 for FIG. 2 is present in the structure suitable for storage and/or transport. When the elements of the biotherapeutic composition, comprising at least moist component 206 and dried bacteria 208 are to be mixed before administration to the subject, handle 112 is grasped and manipulated so as to pierce and/or remove separator 204 as shown with regard to FIG. 3. Hereinafter, “at least partially removed” includes pierced, detached, removed and separated.

In the optional implementation of FIG. 3, handle 112 is optionally pushed or twisted, such that a plunger 300 is pushed against separator 204, causing separator 204 to be at least partially removed or detached. Optionally, plunger 300 features a spring 302 whose tension is increased by twisting handle 112, which tension then forcing plunger 300 against separator 204. Plunger 300 may optionally be present in compartment 202, although optionally plunger 300 may be separated from compartment 202 by a wall (not shown).

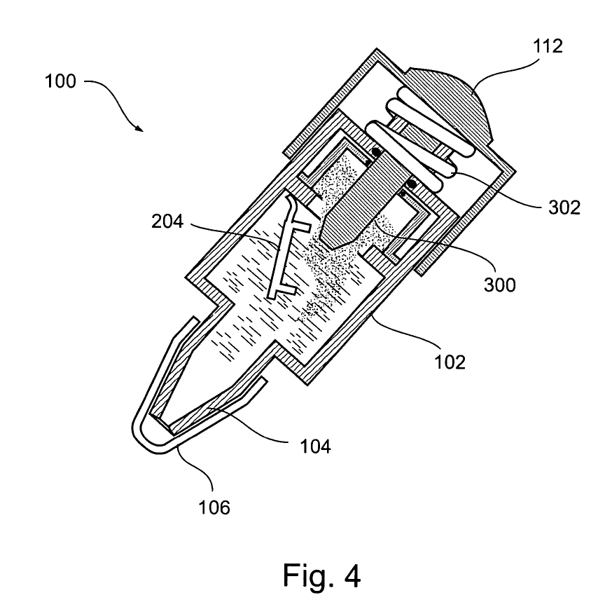

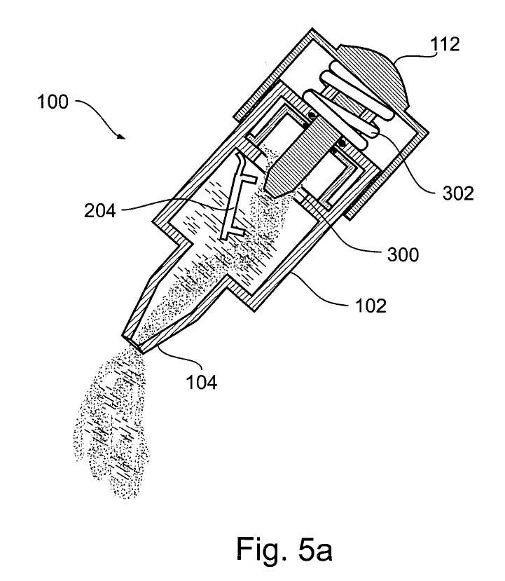

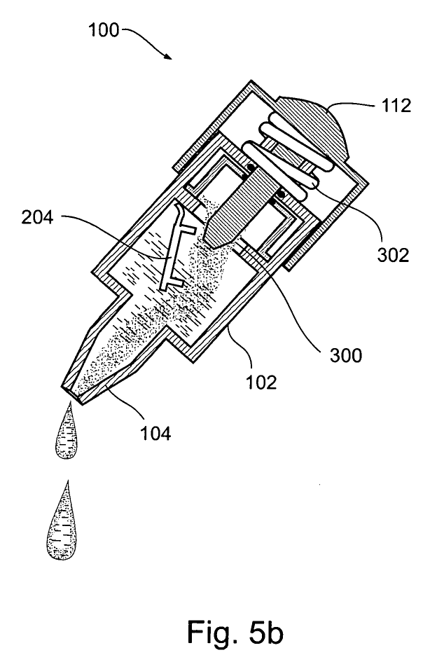

Once separator 204 has been pierced, or at least partially removed or detached, the contents of compartments 200 and 202 may interchange and mix as shown with regard to FIG. 4. When the mixture is ready for administration, the mixture (shown as reference number 500) is preferably allowed to flow out of device 100, after removal of cover 106, as shown with regard to FIGS. 5A and 5B (not all reference numbers are shown for clarity). Depending upon the configuration of an aperture of nozzle 104, mixture 500 may optionally flow out through a large aperture 502, or alternatively may be allowed to drip out in drips through a smaller aperture 504.

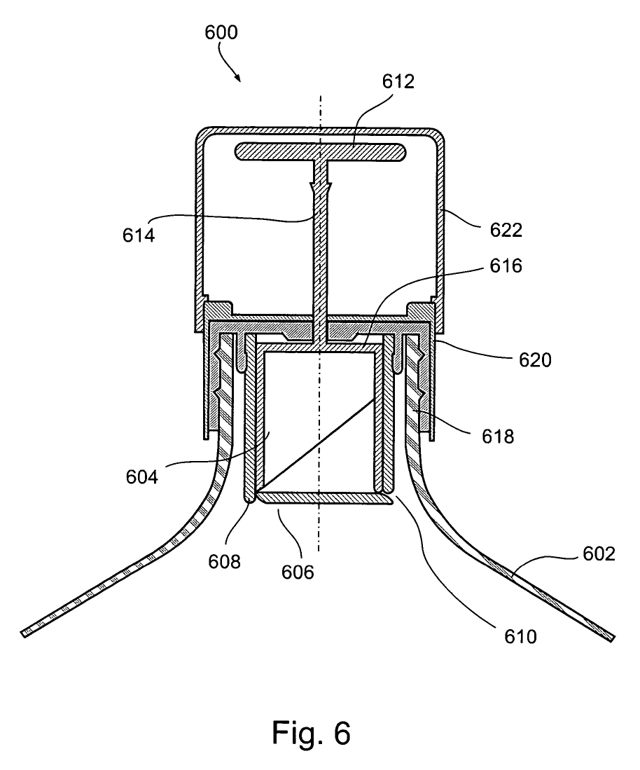

FIG. 6 shows a second embodiment of the dispensing device according to the present invention. It should be noted that in this context, dispensing devices are referred to interchangeably as “dispensers”, “dispensing systems”, or “devices” in this application.

As shown, a dispensing system 600 features a lower container 602, which could optionally be in the form of a bottle for example. Dispensing system 600 also preferably features an upper container 604. One of lower container 602 and upper container 604 preferably stores the moist component, while the other stores the dried bacteria. Preferably, lower container 602 stores the moist component, which could be an aqueous solution for example, while upper container 604 preferably stores the dried bacteria, although this structure could be reversed.

In any case, in order for the contents of upper container 604 to be mixed with the contents of lower container 602, upper container 604 preferably is capable of becoming in communication with lower container 602 through a lower portion 606 of upper container 604. A first part of lower portion 606 preferably features a break-away score 608 for being broken, while a second part of lower portion 606 preferably features a hinge score 610 for hingeably connecting lower portion 606 to upper container 604. When pressure is placed upon break-away score 608, break-away score 608 becomes broken, while lower portion 606 swings on hinge score 610, thereby creating an opening in upper container 604. The contents of upper container 604 may then mix freely with those of lower container 602.

For the preferred embodiment as shown, upper container 604 is preferably suspended above lower container 602, within the neck of lower container 602 as shown. This suspension is optionally and preferably accomplished by attaching upper container 604 to a plunger 612, which features a plunger handle 614 and a plunger portion 616. Plunger portion 616 may optionally form a part of upper container 604 as shown, but in any case is preferably plungeably connected with lower portion 606. Once pressure is placed on plunger portion 616 through plunger handle 614, such pressure preferably causes break-away score 608 to break as described above.

In order to attach upper container 604 and lower container 602, preferably both at least a part of upper container 604 and at least a part of lower container 602 (such as the neck) are attached to an inner cap 618. Plunger 612 (or at least exposed parts such as handle 614) is preferably protected by a protective cap 622, which in turn is preferably snapped onto an outer cap 620 as shown.

When the user wishes to imbibe or ingest the contents of the biotherapeutic formulation, preferably the user removes protective cap 622 and depresses plunger 612 through handle 614. The resultant pressure breaks break-away score 608, causing lower portion 606 to swing on hinge score 610, thereby creating an opening in upper container 604. The contents of upper container 604 may then mix freely with those of lower container 602. Inner cap 618 can then be removed with upper container 604 and so forth, enabling the user to dispense the formulation from lower container 602.

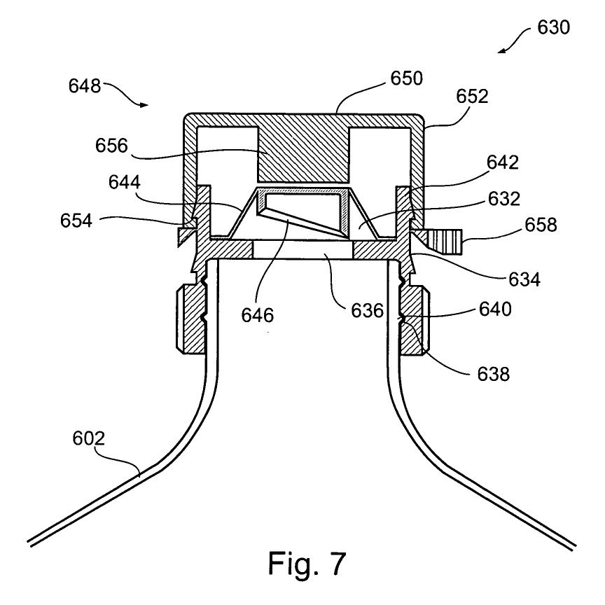

FIG. 7 shows a further alternative embodiment of the dispensing device according to the present invention. Dispensing system 630 differs from dispensing system 600 in that upper container 632 is separated from lower container 602 by an inner cap 634, a portion of which comprises a piercable portion, such as a membrane Cap 634 preferably comprises features for securing cap 634 to an upper portion of lower container 602, such as, for example, grooves 638 positionable over complementary protrusions 640 provided on an outer surface of the neck of lower container 602. Inner cap 634 further preferably comprises a portion to which an outer cap 636 may be attached, such as an upwardly-extending member 642, provided with a lip over which outer cap 636 may be fitted.

Upper container 632 is positioned above cap 634 and comprises flexible walls 644 attached to an upper surface of cap 634, and a piercing component 646 comprising a sharp or pointed object such as a blade, positioned above membrane 636 and associated with walls 644.

Outer cap 648 comprises an upper portion 650 and wall 652. Wall 652 is engaged by upwardly-extending member 642 of cap 634, for example by a lip 654 formed on an outer surface of upwardly-extending member 642. Outer cap 648 further preferably comprises a member 656 extending downward from an inner surface of upper portion 650 of outer cap 648 and positioned above piercing device 646 such that when outer cap 648 is depressed, member 656 exerts pressure on piercing device 646, forcing piercing device 646 to pierce membrane 636 and thus enabling the contents of upper container 632 to enter lower container 602.

Dispensing device 630 is preferably further provided with a safety device for preventing accidental depression of outer cap 648 prior to use, such as, for example, a removable tab 658, positioned directly beneath a lower end of wall 652.

### Example 4

### Methods of Treatment with the Biotherapeutic Composition

As noted above, the biotherapeutic composition of the present invention has been shown to be effective treatments for gastrointestinal diseases and conditions, including but not limited to, microbial infection, IBS and IBD. The following example is an illustration only of a method of treating such a gastrointestinal disease or disorder (or condition in need of treatment), and any other suitable condition with the biotherapeutic composition of the present invention, and is not intended to be limiting.

The method includes preparing the biotherapeitic composition by mixing at least the dried bacteria and the moist component, and also optionally the carrier (if separate) to form a mixture. The moist component may optionally be a liquid or semi-solid formulation. This process may optionally be performed by mixing the elements in a device which holds them in separate compartments until they are to be mixed- in the mixture, as described above. Next, the mixture is preferably allowed to stand for activation of the bacteria. Next, the mixture is administered to a subject to be treated, for example by drinking or otherwise swallowing the mixture.

The mixture of the biotherapeutic composition is administered in a pharmaceutically effective amount according to an effective dosing methodology, preferably until a predefined endpoint is reached, such as the absence of a symptom of a gastrointestinal disease, disorder or condition and any other suitable condition in the subject, or the prevention of the appearance of such a disease, disorder, condition or symptom in the subject.

### Example 5

### Treatment of Diarrhea

This Example is a non-limiting illustrative demonstration of the effect of eliminating episodes of acute diarrhea caused by Salmonella and food intoxications of unknown etiology (including traveler's diarrhea) depending on the quantity of probiotic bacteria administered to a patient per day is shown (dose-dependent efficacy).

A significant number of patients are treated with different therapeutically effective amounts of the mixture of the biotherapeutic composition of the present invention. These quantities are optionally in the range of 10-200 billion live bacteria per day (or another suitable such range), divided into 4-6 doses (or another suitable number of doses). Such administration demonstrates the dose dependent efficacy of he present invention on the symptoms and effects of diarrhea.

### Example 6

### Additional Preparation Embodiment

This Example provides another exemplary, illustrative embodiment of a method of preparing the biotherapeutic composition according to the present invention. The basic method for creating this product may optionally be performed as follows:

Grow E. coli, ATCC 202226 to high-cell density in a fermenter;

Remove and wash the cells, finally resuspending them-in a sucrose-phosphate buffer to a desired density;

Air dry or lyophilize a small aliquot of this suspension in one phase of a biphasic container such that the aliquot contains sufficient sucrose when hydrated to yield a desired concentration in the liquid of the second phase;

Prepare a suitable growth medium, preferably without carbohydrate, in a one-dose aliquot in the second phase, and place in the other compartment;

At a predetermined time before the product is to be used, mix the two compartments and incubate at room temperature for a predetermined amount of time, The amount of growth in the final product will be a function of the amount of sucrose present as a carbon source.

Note: This process as designed is operative with those strains which are able to use sucrose for growth.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are hereini incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A dispenser for dispensing a biotherapeutic composition comprising rapidly activatable bacteria in a dry form, a carrier for containing at least one substance for causing rapid activation of said bacteria and a moist component, the dispenser comprising:
(a) a first compartment for holding said moist component;
(b) a second compartment for holding said bacteria in a dry form;
(c) a separator for separating said first and second compartment, such that when said separator is at least partially removed, said moist component and said dried bacteria are permitted to mix to form a mixture, thereby forming the biotherapeutic composition.

2. The dispenser of claim 1, wherein said separator is partially removable by one of piercing, detaching, removing and separating.

3. The dispenser of claim 1, further comprising a rod having a first end extending out of said body of said dispenser and a second end positioned adjacent to said separator, said rod being depressable within said body, such that manipulation of said rod causes said second end to push against said separator, wherein said separator is pierced or removed.

4. The dispenser of claim 3, wherein said rod is a spring loaded plunger.

5. The device claim 3, wherein said rod is provided with a handle attached to said first end.

6. The device of claim 3, wherein said manipulation comprises one of pushing and twisting.

7. The dispenser of claim 1, wherein one of said first and said second compartments comprises a bottle.

8. The dispenser of claim 1, further comprising a nozzle in communication with said first compartment.

9. The dispenser of claim 8, further provided with a removable cover for sealing of said nozzle.

10. The dispenser of claim 1, wherein said separator is positioned over an aperture between said first compartment and said second compartment, a first part of said separator being connected to a first side of said aperture by a break-away score for being broken, and a second part of said separator being connected to a second side of said aperture by a hinge score, such that when pressure is exerted upon said break-away score, said break-away score becomes broken, and said second part of said separator swings on said hinge score, thereby opening said aperture between said first compartment and said second compartment.

11. The dispenser of claim 1, wherein said separator is a membrane, said membrane being impermeable to said moist component.

12. The dispenser of claim 11, wherein said membrane is removable by piercing.

13. The dispenser of claim 11, wherein said membrane comprises a portion of an inner cap sealing an open upper portion of a lower of said first and said second compartments.

14. The dispenser of claim 13, further comprising a piercing member positioned over said membrane.

15. The dispenser of claim 14, wherein said piercing member comprises an integral part of said upper of said first and said second compartments.

16. The dispenser of claim 14, further comprising a depressible member positioned over said piercing member, such that when said depressible member is depressed, said piercing device is caused to pierce said membrane.

17. The dispenser of claim 16, wherein said depressible member is a plunger.

18. The dispenser of claim 16, wherein said depressible member forms a portion of an outer cap positioned over and engaged by said inner cap.

19. The dispenser of claim 16, further comprising a removable safety device for preventing depression of said depressible member.

20. The dispenser of claim 19, wherein said removable safety device is a pull-tab.

21. The dispenser of claim 20, wherein said pull-tab is positioned beneath a lower edge of said outer cap.

22. A method of preparing a biotherapeutic composition comprising rapidly activatable bacteria in a dry form, a carrier for containing at least one substance for causing rapid activation of said bacteria and a moist component, in at least two separate elements, wherein when said dry form bacteria are selected according to a selection pressure and dried by air drying, said carrier and said moist component are mixed to form a mixture, said bacteria become rapidly activated in said mixture.

23. A biotherapeutic composition, comprising rapidly activatable bacteria in a dry form, a carrier containing calcium alginate for containing at least one substance for causing rapid activation of said bacteria and a moist component, in at least two separate elements, wherein when said dry form bacteria, said carrier and said moist component are mixed to form a mixture, such that said bacteria become rapidly activated in said mixture

24. The method of claim 22, wherein preparation of said dry form bacteria comprises:
(a) providing a seed culture of from about 10⁸ to about 10⁹ of said bacteria;
(b) adding glucose solution at a concentration of about 2.5 g/L to said seed culture to obtain an inoculate;
(c) introducing an amount of said inoculate into a bioreactor;
(d) allowing said inoculate to grow in said bioreactor for at least 24 hours;
(e) cooling said bioreactor to below 10° C. for harvest; and
(f) harvesting contents of said bioreactor by microfiltration.
